(19) United States
(12) Patent Application Publication
(10) Pub. No.: US 2014/0341856 A1
(43) Pub. Date: Nov. 20, 2014

(54) METHOD OF CONTROLLING TOMATO PLANT VIRUSES

(71) Applicant: KUWAIT UNIVERSITY, Safat (KW)

(72) Inventors: NARJES H. DASHTI, SAFAT (KW); MAGDY S. MONTASSER, SAFAT (KW)

(73) Assignee: KUWAIT UNIVERSITY, Safat (KW)

(21) Appl. No.: 13/898,426

(22) Filed: May 20, 2013

(51) Int. Cl. A61K 35/76; A61K 35/74

(52) U.S. Cl. CPC A61K 35/76; A61K 35/74
USPC 424/93.3

(57) ABSTRACT

The method of controlling tomato plant viruses involves inoculation of an uninfected plant with a combination of a cucumber mosaic virus (CMV-KU1) associated with a naturally occurring benign viral satellite RNA with a mixture of two plant growth-promoting rhizobacteria (PGPR) strains, namely, Pseudomonas aeruginosa and Stenotrophomonas rhizophilia, in order to protect plants from the virulent CMV virus while promoting plant growth, yield and fruit quality of the tomato that is lost due to the viral infection. The healthy plant leaves are inoculated with the CMV-KU1 virus at the dicotyledonary stage. Simultaneously, the roots of the tomato plants are inoculated with the PGPR mixture. The satellite RNA component of the combination protects plants against a virulent virus (CMV-16), while the PGPR component compensates for growth, yield, and quality loss of tomato seen in the presence of both CMV-KU1 and CMV-16, in addition to strengthening the protection of plants.

### BACKGROUND OF THE INVENTION

1. Field of the Invention

The present invention relates to a method of controlling tomato plant viruses, and particularly to a method of controlling tomato plant viruses by inoculation with a combination of a cucumber mosaic virus (CMV) associated with a naturally occurring benign viral satellite RNA and two plant growth-promoting rhizobacteria (PGPR) strains, thereby controlling the viruses and improving growth, yield, and fruit quality of tomato plants.

2. Description of the Related Art

Viral diseases pose a serious threat to crop plants, including the tomato plant (Solanum lycopersicon L.). Cucumber mosaic virus (CMV), belonging to the genus Cucumovirus of the Bromoviridae family, is considered to be one of the most economically damaging viruses among field-grown vegetables worldwide. CMV causes systemic mosaic, yellowing, ringspots, deformed fruit and poor fruit set of tomatoes. Some strains of CMV contain viral satellite RNAs, while other strains of CMV are satellite RNA-free strains, such as CMV-16, which causes severe stunting, chlorosis, and malformation of fruits in tomato plants. Viral disease management strategies employed can include the use of non-pathogenic microorganisms or naturally occurring viral satellites that can be used as a biological control agent against such viral infections. These control agents act by enhancing the systemic resistance or acquired resistance of the plants against viruses.

Satellite RNAs are capable of altering the viral phenotype to such an extent that they can modulate, attenuate, or exacerbate the symptoms caused by their cognate helper viruses. Satellite RNAs are small nucleic acids whose nucleotide sequences are unrelated to, but are dependent upon, the viral genome for replication, encapsidation and dispersion; they have a molecular parasitic relationship. Strain KU1 is a mild strain of CMV associated with a benign satellite RNA (345 by long) that induces mosaic symptoms on squash leaves. In tobacco plants, strain KU1 induced mild mosaic on very young leaves but later the plants were symptomless. It is symptomless on tomato and its presence in these plants can be detected only by a slight decrease in vegetative growth and a significant yield loss of about 15-20%.

Plant growth-promoting bacteria (PGPR), such as nitrogen-fixing rhizobacteria that colonize plant rhizospheres, have been studied for their beneficial role in promoting plant growth. The ability to enhance plant growth is limited to specific bacteria and is dependent on (a) their genetic traits, such as motility; (b) chemotaxis to seed and root exudates; (c) production of pili and fimbriae; (d) production of specific cell surface components; (e) ability to use certain cell surface components of root exudates, protein secretion; and (f) quorum sensing. The mechanisms of PGPR to promote plant growth are not fully understood, but are thought to influence the plants, both directly and indirectly. The direct effect of the PGPRs is the promotion of plant growth and is most often observed in the absence of plant pathogens and other competing soil microbes. Indirectly, PGPRs play a vital role in plant protection against plant pathogens, such as viruses and certain fungi. The mechanisms by which they enhance protection include (i) the ability to produce or change the concentration of the plant hormones, such as indoleacetic acid, gibberellic acid, cytokinins, and ethylene; (ii) asymbiotic N₂ fixation; (iii) antagonism against phytopathogenic microorganisms by production of siderophores that chelate iron; (iv) production of β-1,3-glucanase, chitinase, antibiotics and cyanide; and (v) solubilization of mineral phosphates and other nutrients.

The PGPRs used in this work (Pseudomonas aeruginosa and Stenotrophomonas rhizophilia) are well known for their ability to promote plant growth, both individually and in association with one another. Pseudomonas is a diverse group of Gram-negative, aerobic heterotrophic bacteria found in soil, although some are aquatic and some can be found in animals. Individual Pseudomonas strains may have biocontrol activity, plant growth-promoting activity, the ability to induce systemic plant defense responses, or the ability to act as pathogens. Many fluorescent Pseudomonas strains (e.g. P. aeruginosa), which colonize the rhizosphere, exert a protective effect on the roots through the production of in situ antibiotic compounds that promote growth and prevent microbial infections. Stenotrophomonas species, belonging phylogenetically to γ-Proteobacteria, have an important ecological role in the elemental cycle of nature, degradation of xenobiotic compounds, promotion of plant growth and as a biocontrol agent against certain pathogenic fungi. S. rhizophilia is a xylose-utilizing, non-lipolytic, non β-glucosidase-producing Stenotrophomonas species that is capable of growth even at low temperatures (4° C.). These properties offer a great advantage for symbiotic association with plants. S. rhizophilia is also known to have remarkable antifungal activity against plant-pathogenic fungi. This ability to reduce disease, and hence promote plant growth, is largely due to the ability of Stenotrophomonas species to produce siderophores for iron chelation, antibiosis and production of lytic enzymes. S. rhizophila is able to colonize various plant tissues in tomato, sweet pepper, cotton and oilseed rape. In general, the population establishment is higher on the roots and stems than on the leaves. S. rhizophila has been observed as an endophyte of tomato root hairs. The plant growth-promoting effect of S. rhizophila is mostly via the suppression of pathogens and deleterious microbes, which could lead to a better growth environment for the plant.

U.S. Pat. No. 8,138,390, issued Mar. 20, 2012 to Montasser (one of the present inventors), describes the use of the KU1 strain of CMV as a biological control agent for the protection of tomato plants from the KU2 strain of CMV, as well as the potato spindle tuber viroid, fusarium wilt disease, and leaf spotting disease. While effective for these purposes, treatment with the KU1 strain of CMV is typically accompanied by a slight decrease in vegetative growth and a significant yield loss of about 15-20%. It would be desirable to provide a method of protecting tomato plants against other strains of CMV that does not suffer from such side effects. The '390 patent cited above is hereby incorporated by reference in its entirety.

Thus, a method of controlling tomato plant viruses solving the aforementioned problems is desired.

### SUMMARY OF THE INVENTION

The method of controlling tomato plant viruses comprises inoculation of an uninfected plant with a combination of a cucumber mosaic virus (CMV-KU1) associated with a naturally occurring benign viral satellite RNA with a mixture of two plant growth-promoting rhizobacteria (PGPR) strains, namely, Pseudomonas aeruginosa and Stenotrophomonas rhizophilia, in order to protect plants from the virulent CMV virus while promoting plant growth, yield and fruit quality of the tomato that is lost due to the viral infection. The healthy plant leaves are inoculated with the CMV-KU1 virus (described in U.S. Pat. No. 8,138,390) at the dicotyledonary stage. Simultaneously, the roots of the tomato plants are inoculated with the PGPR mixture. The satellite RNA component of the combination protects plants against a virulent virus (CMV-16) that causes severe stunting, leaf curl, yellowing and yield loss in tomato plants. The PGPR component compensates for growth, yield, and quality loss of tomato seen in the presence of both CMV-KU1 and CMV-16, in addition to strengthening the protection of plants.

These and other features of the present invention will become readily apparent upon further review of the following specification and drawings.

### BRIEF DESCRIPTION OF THE DRAWINGS

FIG. 1A is a table showing the effect of PGPRs and viral satellite RNA combinations in lowering disease severity based on Enzyme Linked Immunosorbent Assay (ELISA) and symptom scoring.

FIG. 1B are the footnotes for the footnotes for the table of FIG. 1A.

FIG. 2 is a histogram showing mean disease severity value with different PGPR and viral satellite RNA combinations.

FIG. 3A is a graph showing shoot length with the different treatments and healthy control plants in the presence and absence of CM-16 virus.

FIG. 3B is a graph showing the fresh weight of tomato plants with the different treatments and healthy control plants in the presence and absence of CM-16 virus.

FIG. 3C is a graph showing the dry weight of tomato plants with the different treatments and healthy control plants in the presence and absence of CM-16 virus.

FIG. 3D is a graph showing the fruit yield of tomato plants with the different treatments and healthy control plants in the presence and absence of CM-16 virus.

Similar reference characters denote corresponding features consistently throughout the attached drawings.

### DETAILED DESCRIPTION OF THE PREFERRED EMBODIMENTS

The method of controlling tomato plant viruses comprises inoculation of an uninfected plant with a combination of a cucumber mosaic virus (CMV) associated with a naturally occurring benign viral satellite RNA and two plant growth promoting rhizobacteria (PGPR) strains. The cucumber mosaic virus (CMV) associated with a naturally occurring benign viral satellite RNA used in the present invention is CMV-KU1 that is described in U.S. Pat. No. 8,138,390, which is hereby incorporated by reference in its entirety. The PGPR strains used in the present method are Pseudomonas aeruginosa and Stenotrophomonas rhizophilia. The method protects the plants against harmful viruses, particularly CMV-16, and also results in an improvement in vegetative growth, fruit yield and fruit quality of tomato plants.

### EXAMPLE

Cucumber mosaic viral strain associated with a benign viral satellite RNA (CMV-KU1) was isolated in Kuwait. This virus is symptomless in tomato. The uninfected plants may be inoculated with CMV-KU1 by grinding the tissues of a plant infected with CMV-KU1 virus in a 0.01M potassium phosphate buffer and rubbing the ground CMV-KU1-infected tissues over the leaves of the uninfected plant with a cotton swab. Strain CMV-16, subgroup II, is a Japanese isolate from tomato (kindly provided by H. Sayama, Kikko Foods Corporation, Japan) that contains no detectable viral satellite when maintained in tomato, but causes severe stunting and fruit malformation. This virus was used as a challenge strain. Strains were revived from leaves of old desiccated samples that were available in our Molecular Virology Lab., University of Kuwait. The viral isolates were invigorated by mechanical passage into fresh squash (Cucurbita pepo L.) and tomato (Solanum lycopersicon L.) plants. The infected leaves were ground in neutral 0.01 M potassium phosphate buffer with a mortar and pestle, and the crude sap was used to inoculate the tomato test plants.

Seeds of the tomato cultivar ‘Supermamande’ were surface-sterilized in sodium hypochlorite (2% solution containing 4 ml L⁻¹ Tween 20), and then rinsed several times with distilled water. The seeds were planted by hand into pots washed with sodium hypochlorite solution containing sterilized, soilless growth medium. The soilless growth medium used was prepared by mixing peat moss (Plantaflor) with Perlite in a ratio of 3:1. Following germination, the seedlings were thinned to one plant per pot to ensure better growth. The plants were allowed to acclimatize within the greenhouse for 48 h after reaching the dicotyledonary stage prior to treatment with the PGPR inoculum.

Two strains of PGPR were used in this method, namely, Pseudomonas aeruginosa and Stenotrophomonas rhizophilia. The PGPR strains used were obtained locally from the stock cultures available in our lab. Both the strains used were isolated in previous work from the Vicia faba rhizosphere. Diluted rhizosphere soil suspensions were plated on solid Pseudomonas medium and yeast-mannitol agar for P. aeruginosa and S. rhizophilia, respectively. These were incubated at 30° C. for 7 days, and pure colonies were subcultured. The organisms were identified by the “Deutsche Sammlung von Mikroorganismen and Zellkulturen GmbH (DSMZ)”, Brauschweig, Germany, and by various biochemical tests performed at Kuwait University. The inoculum of the two PGPR strains was prepared by culturing them separately in nutrient broth and incubating at 20-25° C. with constant shaking at 125 rpm. When the cultures reached log phase, each of the strains was adjusted with distilled water at A₄₂₀ to give a cell density of 10⁸ CFU/mL. Equal volumes (1:1) of the two strains were mixed and allowed to stand for approximately one-half hour at room temperature without shaking before use.

Three independent experiments were conducted. Plants were divided into three different treatments: (a) plants treated with satellite virus CMV-KU1 alone (referred to as KU1); (b) plants treated only with PGPR mixture (referred to as PGPR); and (c) plants treated with a combination of CMV-KU1 and the PGPR mixture (referred to as PGPR+KU1). One control treatment without any bacterial or viral inoculation was also included. Treatments were arranged in a randomized complete block design, with 20 plants in each treatment. Each of the three treatments and the control treatment were divided into two subgroups, one challenged with CMV-16, and the other one without CMV-16 at 21 days post-inoculation (dpi) with treatments. Both the PGPR strains and the satellite-associated CMV-KU1 viruses were applied to the plants at the dicotyledonary stage.

The PGPRs were applied to the base of the plants close to the roots to ensure better colonization. The volume of PGPR inoculated per plant was 10 mL, each containing a cell density of 10⁸ CFU /mL. The virus was applied onto the plants by mechanical sap transmission. The leaves of the tomato plants were dusted with abrasive carborundum, and then rubbed with infected leaf sap ground in 0.01 M phosphate buffer using a sterile cotton swab. Plants were maintained under greenhouse conditions with alternating 16 h light and 8 h dark periods. The temperature regime was maintained at 25° C. Fertilization was carried out every alternate day using sterile Hoagland's solution. Perforated pots were used to ensure proper drainage of excess amount of solutions. Twenty-eight days after inoculation, the plants were repotted into bigger pots. Plants were scored for symptoms at 28, 35 and 42 days post-inoculation with the biological treatments.

Forty-two days post-inoculation with the different treatments, the plants were harvested by following the procedure described by Radwan et al. (J. Phytoremed. Sep. 1-11, 2007). Plants were carefully dislodged from the soil, taking special care not to sever the fine root hairs. All soil that was not part of the rhizosphere (i.e., not attached or close to the roots) was carefully removed by gentle tapping. After washing the roots, the heights, fresh weights, and fruit yield were measured. The plants were then placed in paper bags and kept in an oven for 2-3 days for dry weight determination. Approximately 10 g of the soil attached to the roots of each treatment was transferred into 90 mL of sterile distilled water. This was shaken for 10-20 in, after which 1 mL aliquot from this mixture was taken, serially diluted (10-fold), and finally plated by spreading on nutrient agar. Incubation was done for 48 h at 20-25° C. Eight dilutions were prepared per treatment, with two replicates for each dilution. Control plates were also incubated.

The CMV-16 accumulation in the foliar tissue of untreated and treated test plants was investigated using the indirect ELISA method. Each plant was sampled at the end of 42 days by collection of three terminal leaflets from three young non-inoculated leaves. For normalizing the samples for ELISA, 0.5 g of plant tissue was mixed with 10×(w/v) coating buffer (15 mM sodium carbonate, 35 mM sodium bicarbonate, pH 9.6, containing 2% polyvinyl pyrrolidone: CB-PVP), then homogenized using a mortar and pestle for sap extraction. Extracted crude sap was filtered through cheesecloth and centrifuged at 6,000 g for 2 min. The clarified extract was pipetted into wells of polystyrene microtiter plates. The antigen solution was stored overnight at 4° C. in glass tubes before the coating incubation for 2-3 h, or incubated directly in microwell plates, either at 4° C. overnight, or at 37° C. for 3 h. After 3 washes for 3 min each with phosphate-buffered saline (PBS) containing 0.5% Tween-20 (PBS-T), the plates were blocked by incubation in 1% bovine serum albumin (BSA) in PBS for 30-60 min. The blocking solution was replaced by an appropriate dilution of a specific monoclonal antibody against CMV-16 (Affinity Bioreagents, NJ, USA) that was incubated at 37° C. for 60 min. This was followed by 3 washes with PBS-T and the addition of goat anti-mouse alkaline phosphatase conjugate diluted in PBS buffer (1:1000), after which the solution was incubated at 37° C. for 3-4 h. After 3 washes with PBS-T, p-nitrophenyl phosphate was added in the substrate buffer (pH 9.8). The absorbance was measured at 405 nm, 15-60 min after the addition of the substrate, using a Biotek Model EL307 (Burlington, Vt.) or a Dynatech MR700 ELISA Reader (Bio-Rad Laboratories, Inc. U.K.). Values that exceeded twice that of the untreated/healthy samples and/or the buffer controls were considered positive. Based on the ELISA values, the percentage infection of plants in each treatment was also calculated.

Based on the visibility of symptoms appearing on the plants, the plants were scored from a scale of 0 to 10, where 0=no symptoms and 10=severe symptoms. A logistic model was fitted to assess disease intensity, area under the disease progression curve, and disease prevention. This model is given by the following equations. Disease Intensity=100(Σ sn/SN), where s is the disease score, S is the highest s grade, n is number of plants with the same s value, and N is total number of test plants indexed. Area under disease progress curves (AUDPC) was calculated using the formula: Σ (0.5) (Yᵢ+Yᵢ₊₁) (Tᵢ+Tᵢ₊₁) where Y=disease severity at time T, and i=the time of the assessment in days. Disease prevention was calculated using the formula 100([C−T]/C), where C=disease intensity of control plants inoculated only with CMV-16 and T=disease intensity of three treatments plants challenged with CMV-16. Analysis of variance (ANOVA) at P=0.05 was performed on all the data using the SPSS (Statistical Package for Social sciences)—PASW statistics 18 software, and the means were separated with Duncan's Multiple Range Test (DMRT) using PASW statistics 18 and the Michigan University Statistical Package (MSTATC) software. The Graphs were constructed using the Slidewrite program. The standard error was calculated by dividing sample standard deviation values with the square root of the total number of samples in each treatment. The error bars were drawn on the graphs based on the standard error calculation. The statistical analysis was done together for all three independent experiments.

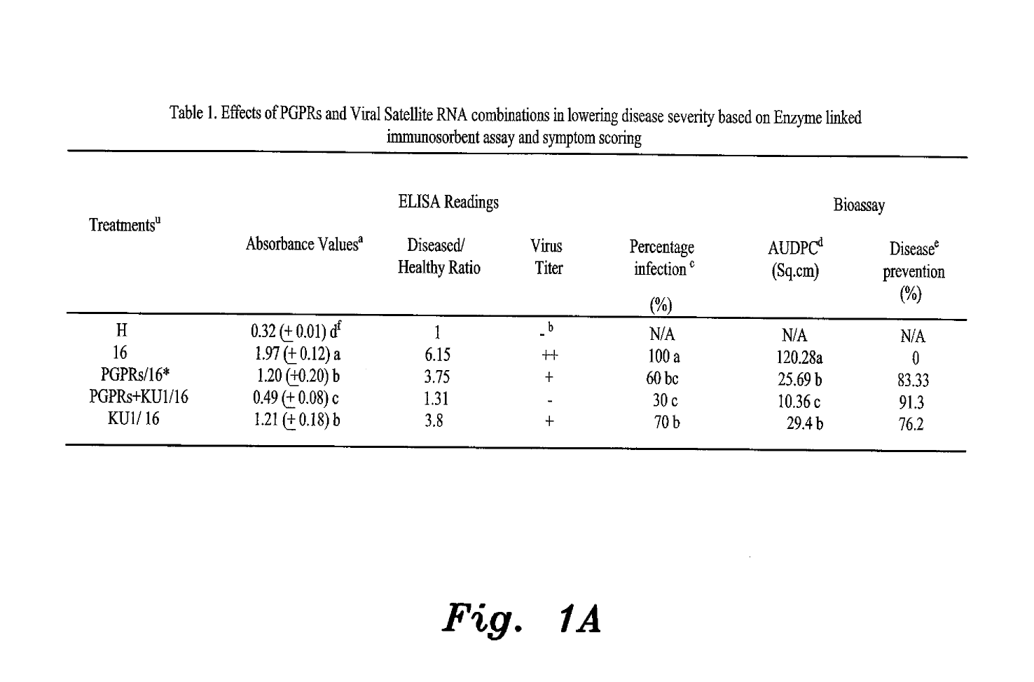

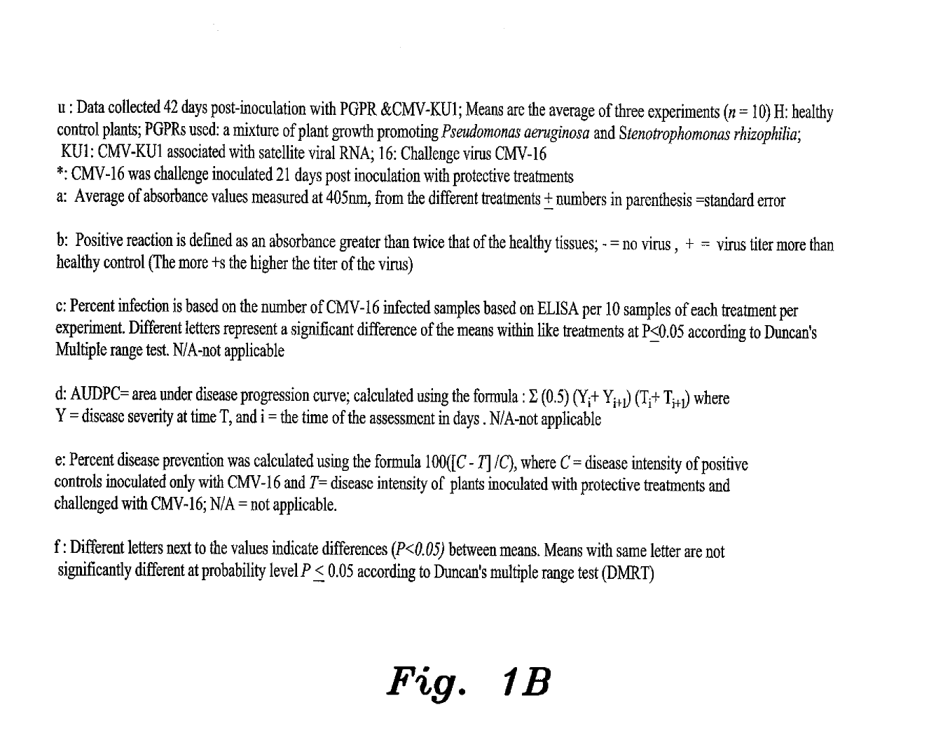

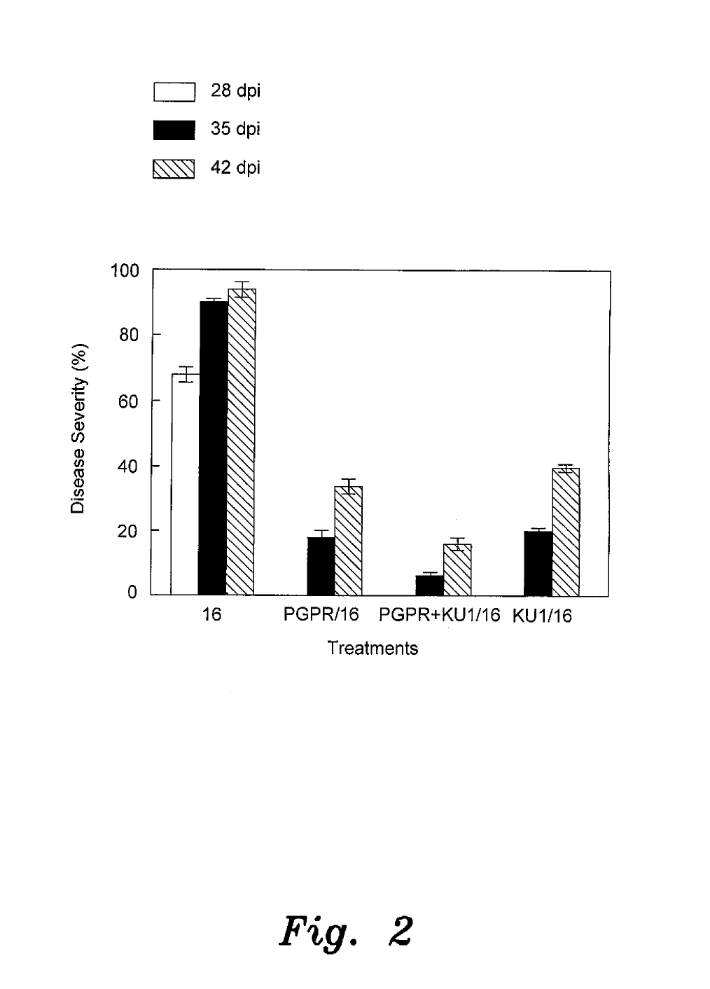

The efficacy of the combination of CMV-KU1 and the two PGPR strains in enhancing protection was determined based on visual observation for the appearance of virus symptoms and by serological analysis (FIGS. 1A, 1B and 2). All PGPR-treated plants showed a lower disease severity rating when compared to the control plants challenged with CMV-16. The protection against CMV-16 infection was highest for plants treated with PGPR and CMV-KU1 in combination, and lowest for those treated with CMV-KU1 alone. The disease-reducing capacity of the combination was 91.3%. In comparison, the plants treated with either PGPR or CMV-KU1 individually reduced the disease by 83.3% and 76.2%, respectively (FIG. 1A-1B).

The control plants challenged with CMV-16 (referred to as 16 or positive control in this invention) showed severe stunting, outbreak of mosaic symptoms, leaf curling, and loss of vigor as a result of infection. The stunting and the mosaic symptoms were observed in all of the positive controls within 7 days of being challenged with CMV-16. The disease severity value of the positive control was calculated to be at 94% by the third week after challenge with CMV-16 (FIG. 2), indicating a high rate of disease incidence and progression.

The appearance of symptoms and severity of the disease in the treatment KU1 when challenged with CMV-16 was delayed, compared to the positive control. The disease severity ratings at 28, 35 and 42 dpi are shown in FIG. 2. The plants in this treatment were already slightly stunted compared to control plants not challenged with CMV-16 virus (referred to as H or Healthy controls) due to the presence of CMV-KU1. This difference was even more pronounced when challenged with the CMV-16 virus. Plants treated with a PGPR mixture (PGPR/16 & PGPR+KU1/16) had a comparatively lower disease severity rating compared to those treated with CMV-KU1 alone (FIG. 2). There was also a delay in the onset of symptoms. At 35 dpi, mosaic symptoms began appearing on the plants, although not as severe as the CMV-16 positive controls or CMV-KU1 treated plants. There was, however, a decline in plant height and vigor compared to standard treatment (PGPR) without CMV-16 virus infection. These values, however, were comparable to the healthy control plants without any protective treatments. AUDPC values of the different treatments are shown in FIG. 1A-1B.

CMV accumulation, determined by ELISA, showed that the absorbance values at 405 nm observed at 42 dpi were significantly lower in treated plants compared to the positive controls (FIG. 1A-1B). The absorbance value was 0.32 for the healthy controls. Absorbance values that exceed twice that of the healthy values are considered positive. All the treatments not challenged with CMV-16 were less than twice the absorbance value of the healthy control, and therefore were considered as negative. For treatments challenged with CMV-16, only PGPR and CMV-KU1 in combination showed absorbance values below the threshold value. The other two treatments showed positive reaction, the degree of infection indicated by a ‘+’ sign (FIG. 1A-1B). The percentage infection based on the absorbance value of all samples higher than the threshold value per treatment indicated that the percentage of plants infected when protected by a combination of PGPRs and the viral satellite CMV-KU1 were lower than those protected by one of them alone (FIG. 1A-1B). Analysis for disease severity values, AUDPC values, and absorbance values at 405 nm were highly significant, at P≦0.05 (FIGS. 1A, 1B and 2).

The use of viral satellite RNA (CMV-KU1) and PGPRs individually for plant protection against viruses and promoting growth has been previously reported by a number of scientists. The PGPR treatments, when applied to seeds of tomato and cucumber, significantly reduced the AUDPC values of CMV-inoculated plants compared with nonbacterized CMV-inoculated controls. Also, PGPR-mediated induced resistance was previously reported against Tobacco necrosis virus (TNV) and Tobacco mosaic virus (TMV) and Tomato mottle virus (ToMoV). However, their combined effect in preventing disease has not been researched so far. The results of the present method revealed that PGPR-mediated protection, in combination with the viral satellite CMV-KU1, has a positive effect on protection of plants against viral infection, and that the combined effect of the protective strain of CMV KU1 and the PGPRs progressively reduced stunting caused by CMV-16 to a great extent. Plants of all three treatments exhibited a delayed response to infection compared to the positive controls. The disease reduction percentage, disease severity values and AUDPC values indicated that the combination of the PGPRs and CMV-KU1 enhanced protection of the tomato test plants by about 10-20% (FIGS. 1A, 1B and 2) compared to the individual protection conferred by either the satellite viral or PGPR alone. From the ELISA results, it is clear that plants, when protected with PGPR alone, did not reduce the virus titer. Even though the symptoms of the severe virus are attenuated due to high vegetative growth, the virus accumulation in the tissues, based on the ELISA results, is still comparatively high. Similarly, the loss of vegetative growth in plants treated with the satellite-associated helper virus (CMV-KU1) alone may depreciate the protective capability of benign satellite RNA. This is indicated by a high accumulation of CMV-16 in the tissues of these plants (FIG. 1A-1B). When PGPRs were combined with CMV-KU1, virus titers were brought down to values equivalent to those of the healthy control. The presence of CMV-KU1 competitively prevents CMV-16 replication in the foliar tissues, while the presence of the PGPRs in the roots enhances the overall natural defenses of the plants, thus providing double-fold protection against the CMV-16 virus. This is the added advantage compared to using each of them alone. PGPR-mediated biocontrol can be extended to foliar and systemic diseases, even when the PGPRs are applied to seeds and roots.

We observed that both PGPRs and CMV-KU1 strain associated with the viral satellite RNA require a minimum of three weeks to establish themselves and provide protection (data not shown). This allows the PGPRs to successfully colonize the roots and the CMV-KU1 to spread and multiply in the leaves. PGPR and satellite virus-treated plants challenged with severe CMV-16 viral strain without providing sufficient time to establish themselves were infected as severely as the positive controls. The enhanced plant growth due to the presence of PGPRs was also found to be an added advantage for the enhanced protective effect. The protective effect of the PGPRs will therefore vary from strain to strain, depending on their ability to promote growth, either directly or indirectly.

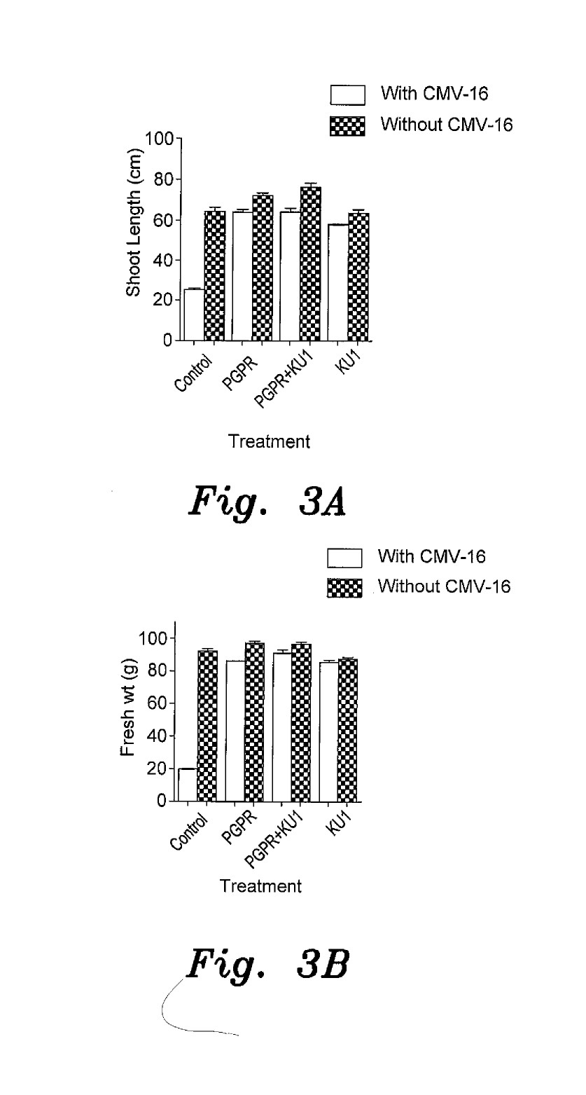

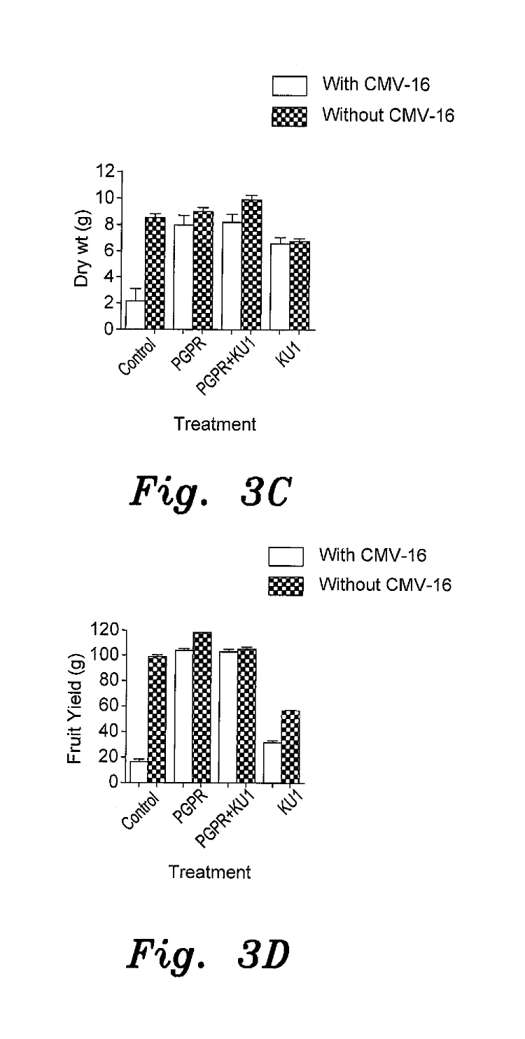

The ability of the PGPRs to compensate for the vigor and yield loss was determined by the mean differences in the shoot length, weight (fresh and dry) and the fruit yield. There was a significant improvement in the height, weights and the fruit yield when PGPRs were added to CMV-KU1. The growth parameters of the different treatments and healthy control plants are shown in FIGS. 3A-3D. The growth of plants treated with a combination of PGPRs and CMV-KU1 in the absence of CMV-16 was much higher than that of the healthy controls. In the presence of the virus, there was a small decline in growth. Prior to the application of the treatments, the growth of all the plants was similar to each other. After the application of the protective treatments one week after germination, all the plants receiving PGPRs showed improved growth, root and leaf development, while those treated with the CMV-KU1 without the addition of PGPR showed vegetative stunting. By the third week, this growth difference was very distinct. The leaf area of the PGPR-treated plants was greater compared to the healthy controls without any inoculation and treatments containing CMV-KU 1. Similarly, root lengths of the plants with PGPRs were also longer. The application of the challenge virus caused a reduction in the vegetative growth in all the treatments. However, for treatments with PGPR, this decline in growth was not less than healthy plants of the same age without infection.

The shoot height, fresh weight and dry weight (FIGS. 3A-3C) showed some differences. The most pronounced differences between treatments were in the yield. Treatments receiving PGPR showed a significant increase in fruit yield (FIG. 3D). These values were significantly higher than the healthy controls. For treatments with CMV-KU1 alone, the average fruit yield was lower than the healthy control (FIG. 3D). The flower abscissions were lower for all plants treated with a protective biological control agent, whether PGPR, CMV-KU1, or the combination, compared to the positive control. However, flower drop was higher in treatments receiving only CMV-KU1. The fruit size and setting was also better for plants receiving PGPR treatments. The average fruit size was very small for positive controls challenged only with CMV-16, compared to the other treatments.

The higher average fruit yield in treatments containing PGPR mixtures indicates that PGPRs not only promoted plant growth, but also promoted fruit yield as well. The beneficial ability of the PGPR, however, may vary with the PGPR strains used and its mechanism in promoting plant growth. The PGPRs used in this method have successfully been tested on different plants, including tomato, cucumber and peppers by other investigators. Both of the strains used were indigenous and were capable of degrading hydrocarbons, thereby enhancing nutrient availability in the soil. They also aid in increased nitrogen absorption by plants. The success of PGPRs is also dependent on the compatibility of the individual PGPR strains used in an inoculum mixture. Adding PGPRs in compatible mixtures has been found to be more successful than using individual strains. The ability of the PGPRs to increase yield and size of fruits to values higher than healthy controls may have economic benefits.

It is to be understood that the present invention is not limited to the embodiments described above, but encompasses any and all embodiments within the scope of the following claims.

We claim:

1. A method of protecting a tomato plant against harmful cucumber mosaic virus (CMV) infection, comprising the steps of:
inoculating an uninfected plant with a therapeutically effective dose of an isolated strain of CMV known as CMV-KU1;
simultaneously inoculating the uninfected plant with a mixture of cultures of two plant growth-promoting rhizobacteria (PGPR) strains in equal parts by volume.

2. The method of protecting a tomato plant according to claim 1, wherein the two PGPR strains are Pseudomonas aeruginosa and Stenotrophomonas rhizophilia.

3. The method of protecting a tomato plant according to claim 1, wherein the cultures of Pseudomonas aeruginosa and Stenotrophomonas rhizophilia each have a cell density of 10⁸ CFU/mL.

4. The method of protecting a tomato plant according to claim 1, wherein said step of inoculating an uninfected plant with CMV-KU1 comprises inoculating the leaves of the uninfected plant with the CMV-KU1 virus.

5. The method of protecting a tomato plant according to claim 4, further comprising the step of inoculating the roots of the uninfected plant with the PGPR mixture.

6. The method of protecting a tomato plant according to claim 4, wherein said step of inoculating the leaves of the uninfected plant with the CMV-KU1 virus comprises inoculating the leaves of the uninfected plant at the dicotyledonary stage.

7. The method of protecting a tomato plant according to claim 1, wherein said step of inoculating an uninfected plant with CMV-KU1 comprises the steps of:
grinding the tissues of a plant infected with CMV-KU1 virus in a 0.01M potassium phosphate buffer; and
rubbing the ground CMV-KU1-infected tissues over the leaves of the uninfected plant with a cotton swab.

8. The method of protecting a tomato plant according to claim 1, wherein the harmful cucumber mosaic virus (CMV) infection comprises an infection caused by CMV-16 virus.
